(19) United States
(12) Patent Application Publication
(10) Pub. No.: US 2018/0110993 A1
(43) Pub. Date: Apr. 26, 2018

(54) CONNECTION PIN AND FEEDTHROUGH

(71) Applicant: BIOTRONIK SE & CO. KG, BERLIN (DE)

(72) Inventors: HELLA EIGENBROD, WENDELSTEIN (DE); LILLI FRIES, STEIN (DE)

(21) Appl. No.: 15/725,316

(22) Filed: Oct. 5, 2017

(30) Foreign Application Priority Data
Oct. 21, 2016 (EP) 16195002.7

(51) Int. Cl. A61N 1/375; H01R 4/02; H01R 4/58; H01B 1/02

(52) U.S. Cl. CPC A61N 1/3754; H01R 4/02; H01R 2201/12; H01B 1/02; A61N 1/37512; H01R 4/58

(57) ABSTRACT

A connection pin for electrical connection of a cable mount. The connection pin has a thickened end for an integrally bonded connection to a conductor surface of the cable mount. At least part of the surface of the thickened end has a solderable coating with at least two functionally differentiated sub-layers arranged one above the other. One of the sub-layers is a solder connection layer with an anti-oxidation layer provided above the solder connection layer and/or an adhesion promoter layer provided beneath the solder connection layer.

### CROSS-REFERENCE TO RELATED APPLICATION

This application claims the benefit, under 35 U.S.C. § 119, of German patent application EP 16195002.7, filed Oct. 21, 2016; the prior application is herewith incorporated by reference in its entirety.

### TECHNICAL FIELD

The invention relates to a connection pin for the electrical connection of a cable mount, which is intended for integrally bonded connection to a conductor surface of the cable mount. The invention also relates to a feedthrough of an implantable electro-medical device (IMD).

### BACKGROUND OF THE INVENTION

Most implantable medical electronic devices (IMDs) of practical significance are intended to deliver electrical pulses to excitable body tissue via suitably placed electrodes. Many devices can also selectively measure electrical pulses and stimuli in the patient's body and can record or evaluate said signals over a relatively long period of time in order to select individually tailored therapy and in order to monitor the success of the treatment in vivo.

In order to perform these functions, electronic/electrical function units for generating and measuring the pulses and for suitably controlling the pulse generation are housed in the housing of the device, and electrodes or connections are provided directly externally on the device for at least one electrode lead, in the distal end portion of which the electrodes are attached to the tissue for pulse transmission. The electronic/electrical function units in the interior of the device are to be connected to the outer electrodes or electrode lead connections in such a way that a completely and permanently reliable function is ensured under the specific conditions of the implanted state.

This task is performed by what are known as feedthroughs, which have been the subject of numerous and very varied developments. The purpose of a feedthrough lies in guiding the electrical signals through the hermetically sealed housing and thus enabling electrical contact of the electronics in the hermetically tight housing with the electrodes in the patient's body. In many feedthroughs of this type, connection pins are used, which are contacted in the interior of the device with the printed circuit board disposed there or a similar cable mount and guide the signals through the housing.

U.S. Pat. No. 7,747,321 B2 discloses, as shown in FIG. 1, a cardiac pacemaker 1 with a pacemaker housing 3 and a head part (header) 5, in the interior of which, besides other electronic components, there is also arranged a printed circuit board (PCB) 7 and, connected to the lead connection thereof (not shown) arranged in the header, an electrode lead 9. A feedthrough 11 provided between the device housing 3 and header 5 comprises a plurality of connection pins 13. The connection pins are plugged at one end through a corresponding bore in the printed circuit board and are soldered thereto. They comprise a wire core, for example made of tantalum, niobium, titanium, molybdenum or copper, and a sheathing that is resistant to oxidation and that is made of a biocompatible material, for example gold, platinum, titanium or the like.

In known feedthrough designs, pin-and-plate constructions are used as connection elements, in which case an elongate connection element (pin) penetrates an insulation body of the feedthrough and is used for welding to a header of the IMD, whereas a plate placed on the housing-side end of the pin produces the contact to the device electronics (printed circuit board). The pin can be made of materials such as Nb or Ptlr, and the plate can consist of Cu or Ni; the pin is brazed in the insulation body, and the plate is usually also brazed to the end of the pin or is connected thereto via a laser spot weld. The plate is usually protected against oxidation by a solder layer (tin plating) or by a thin Pd layer.

U.S. Pat. No. 7,340,305 B2 discloses a corresponding feedthrough of an IMD in which connection pins are used that have a core not consisting of costly platinum, platinum/iridium or palladium, and a conductive coating. The coating makes it possible on the one hand to control or to limit an oxidation of the connection pin under the conditions of use of an implanted device and on the other hand makes it possible to connect the connection pin by a soldering method to a printed circuit board. U.S. Patent application US 2011/0303458 A1 also discloses connection pins with a partially multi-layer design and use thereof in feedthroughs of IMDs.

United States patent application US 2016/0104947 A1 discloses a further multi-part connection element design of a feedthrough of an IMD, in which a cap made of a harder material than the pin is fitted onto the connection pin instead of a plate and is then coated in an immersion bath with soldering agent.

In the course of electronics miniaturization and development, the soldering technique has changed rapidly in recent years. Manual component placement processes have been increasingly ousted by fully automatic pick-and-place machines, and through-hole technology (THT) has gradually been replaced by surface-mount technology (SMT). This has enabled smaller, more compact circuits, and thus has also led gradually to smaller IMDS with greater patient compatibility. In order to achieve the many advantages of SMT, feedthroughs must meet the requirements of a surface-mounted device (SMD).

The above-mentioned prior art solutions require a high mounting and overall manufacturing effort. Components are created that are joined to the feedthrough after or during the brazing. The additional component placement effort is not insignificant. It can cause the manufacturing costs for the additional components to exceed their material value many times over. The additional components are mostly very small and delicate (typically <1 mm) and are therefore difficult to place and orientate. Separate manufacturing aids for mounting the additional components are also required. The components must be subjected to extended storage and examination before the mounting and require additional batch management and checking.

Rejects are potentially produced by the additional joining process between pin and add-on element (plate). If the joining process of the pin from components is integrated with the joining process of the feedthrough, an additional sorting check must be performed, in which the process of the pin joining is assessed. After the joining, the connection point must be examined for adhesion, sufficient stability and service life. In principle, a tilting and an offset of the component parts of the pin and, as a result, an offset at the time of placement on the printed circuit board can occur. This must be determined in a separate checking step and must be avoided and checked at the time of feedthrough mounting.

### BRIEF SUMMARY OF THE INVENTION

The object of the invention is to provide a connection pin that is improved compared to known arrangements and that in particular can be produced and processed economically and yet is still of high quality, and the use of which enables a reduced checking and mounting effort with the mounting (SMT) of feedthroughs.

With the above and other objects in view there is provided, in accordance with the invention, a connection pin for electrical connection of a cable mount, the connection pin comprising:

a thickened end for an integrally bonded connection to a conductor surface of the cable mount;

at least a portion of a surface of said thickened end having a solderable coating with at least two functionally differentiated sub-layers arranged above one another, wherein one of said sub-layers is a solder connection layer with an anti-oxidation layer provided above said solder connection layer and/or an adhesion promoter layer provided beneath said solder connection layer.

In other words, according to the invention the connection pin has a thickened end and at least part of the surface of the thickened end has a solderable coating with at least two functionally differentiated sub-layers arranged one above the other, of which the uppermost is an anti-oxidation layer, below which the solder connection layer and/or an adhesion promoter layer is provided. As a result, the proposed connection pin can be joined on a conventional SMD printed circuit board or a similar cable mount by means of an established soldering method, wherein the soldering method is compatible with established processes and materials and provides a connection that is stable in the long term. The multi-part coating of the connection pin enables the optimization of the quality/cost ratio, storage capability and handling properties.

Compared to established designs that include multi-part connection pins, the following specific advantages are provided:
integration of a number of components in one component
more economical production without redesign of the products or product redevelopment
fewer rejects
lower component placement effort, resulting in lower costs
greater measurement and positioning accuracy.

In an expedient embodiment of the invention, the coating is constructed so that it comprises, on a connection pin body, the adhesion promoter layer and at least directly there above the solder connection layer, and at least directly there above the oxidation layer.

Generally, the coating can cover the entire surface of the thickened end, which enables a technically particularly simple production of the coating. On the other hand, it can be expedient that the coating covers only the base and optionally the periphery or portions of the periphery of the nail head-like thickened end. A coating can lead to the advantageous embodiment of a direct soldering of the connection pin with the cable mount or also bonding of connection wires to the connection pin.

The solder connection layer can contain at least one precious metal or one of the metals nickel, copper, silver, tin or an alloy with at least one of these metals. Typical layer thicknesses are a few micrometers. Related metals can also be used, for example one or more of the elements iridium, rhodium or ruthenium. It goes without saying that a wide range of alloys of at least two of the elements mentioned here, advantageously in particular commercially relatively economically available alloys, can also be used in the embodiment of the invention. In particular, copper or titanium, but also biocompatible elements such as tantalum, niobium or molybdenum and also again alloys thereof are possible as material of the untreated connection pin or the pin main body. The adhesion promoter layer can comprise in particular one of the metals palladium, tungsten, platinum or tantalum or one of the compounds niobium nitride, nickel nitride, titanium nitride, copper nitride or the like.

Alternatively, the connection pin can be made of an economical substrate (for example copper, Constantan, nickel). In the embodiment in which the material is not a biocompatible material, the pin is coated after the shaping method with a biocompatible material (for example titanium, tantalum, niobium, platinum, palladium or alloys thereof). The coating must comprise at least one part which is not disposed inside the subsequent IMD housing.

In a further embodiment which in particular lies in the context of the aforementioned aspect of the invention, a sub-layer of the coating is formed as an anti-oxidation layer, and this comprises in particular at least one of the precious metals, such as gold, platinum, palladium or the like. The anti-oxidation layer can be applied above or below the solderable coating. It may also be expedient to produce a plurality of anti-oxidation layers applied one above the other and to combine these or layer these one above the other so as to achieve a particularly high service life of the feedthrough formed by such a connection pin.

In accordance with a technical embodiment of the invention the coating or at least one layer thereof is embodied as a galvanic coating or as a thin layer produced by a vacuum-coating method. Expedient layer thicknesses lie between 0.01 μm (for a thin-film method) and 10 μm (for a galvanic method). Both embodiments have specific advantages and disadvantages; for example the layer coating in the case of galvanic coating is very selective, however the layer is often contaminated with foreign materials from the galvanic bath. The sputtering under vacuum occurs on all sides and with very high purity.

In order to protect the coating against impurity, contamination and oxidation, the coating can be sealed on the pin until further processing. For this purpose, a polymer or an organic protective film can be used (OSP—organic surface protection). Known protective films (for example Glicoat®, ENTEK+®) can be completely or selectively applied to the solderable layer or the entire pin. Typical layer thicknesses are 0.2 μm to 0.6 μm and for example contain substituted imidazoles and/or triazoles. The protective film prevents the oxidation of the base material during storage, typically for several months, and pyrolyses directly before or during the soldering process. Combustion residues on the circuit boards can be removed without residue during the established washing process in automated facilities. Further possibilities lie in the coating by an element of the precious metal groups.

In further embodiments of the invention, the thickened end portion is formed rotationally symmetrically at the end of the connection pin substantially in the form of a nail head. This embodiment can be produced very easily and economically with long-established upsetting methods and, depending on the specific embodiment, offers the possibility of a particularly simple adaptation of the end face of the connection pin to the base area available for the connection to the corresponding conducting track.

The thickened pin end (nail head) can be produced by forming methods, and for this purpose a drawn wire made of Nb/PtIr/Ni/Ta/Cu or the like is cut to the desired length. A head is then formed at one end. The shape of the nail head can be produced furthermore by forming methods, i.e. by casting or alternative manufacturing methods, or by material-removing shaping.

In further embodiments the shaping at the end of the pin is adapted to the contact face (land pattern) of the printed circuit board (PCB). Here, known and frequently used base area shapes are rectangles, hexagons and octagons or other polygons. In order to be able to ensure simple producability, the corners of the forming tool must be rounded so that the pin can be demolded after the forming process. The contact area between feedthrough pin and printed circuit board is increased, whereby the transition resistance can be reduced and the load-bearing capacity of the joint between pin and circuit board can be increased.

In further embodiments a portion that is conical or semi-conical in particular and that tapers towards the outermost end, or also a balloon-like “attachment,” is shaped at the end of the connection pin. Such an additional geometry on the underside of the pin which takes on the function of a bearing face against the circuit board is advantageous for the following reason: The pin should not terminate over its entire area with the circuit board during the soldering process. A defined solder gap is formed between the pin and circuit board, into which gap liquid solder can penetrate or in which gap solder paste is not completely displaced or remains at defined points as the feedthrough the other of the component parts are fitted in position in the solder paste.

It may also be advantageous to make the pin ends pointed or sharp-edged so that these can be centered or fixed by defined pressing in the substrate material of the printed circuit board (PCB). It is thus ensured that the feedthrough does not float or slip in the solder paste as it is passed through the reflow furnace. Soldering defects such as side overhangs or spikes can thus be effectively reduced. The process can thus be performed with usual process parameters, even if the center of gravity or the moment of inertia of the feedthrough is atypical for SMD component parts due to its design.

Other features which are considered as characteristic for the invention are set forth in the appended claims.

Although the invention is illustrated and described herein as embodied in a connection pin and feedthrough, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 shows a schematic illustration of a conventional cardiac pacemaker, and

FIGS. 2 to 7 each show longitudinal sectional illustrations of connection pins according to exemplary embodiments of the invention.

### DETAILED DESCRIPTION OF THE INVENTION

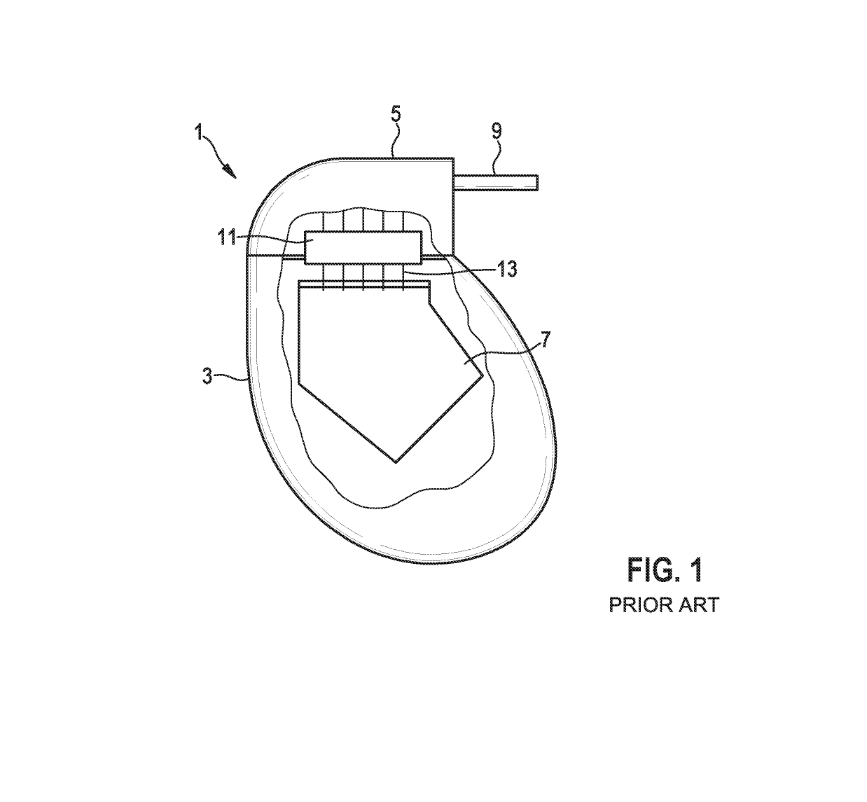

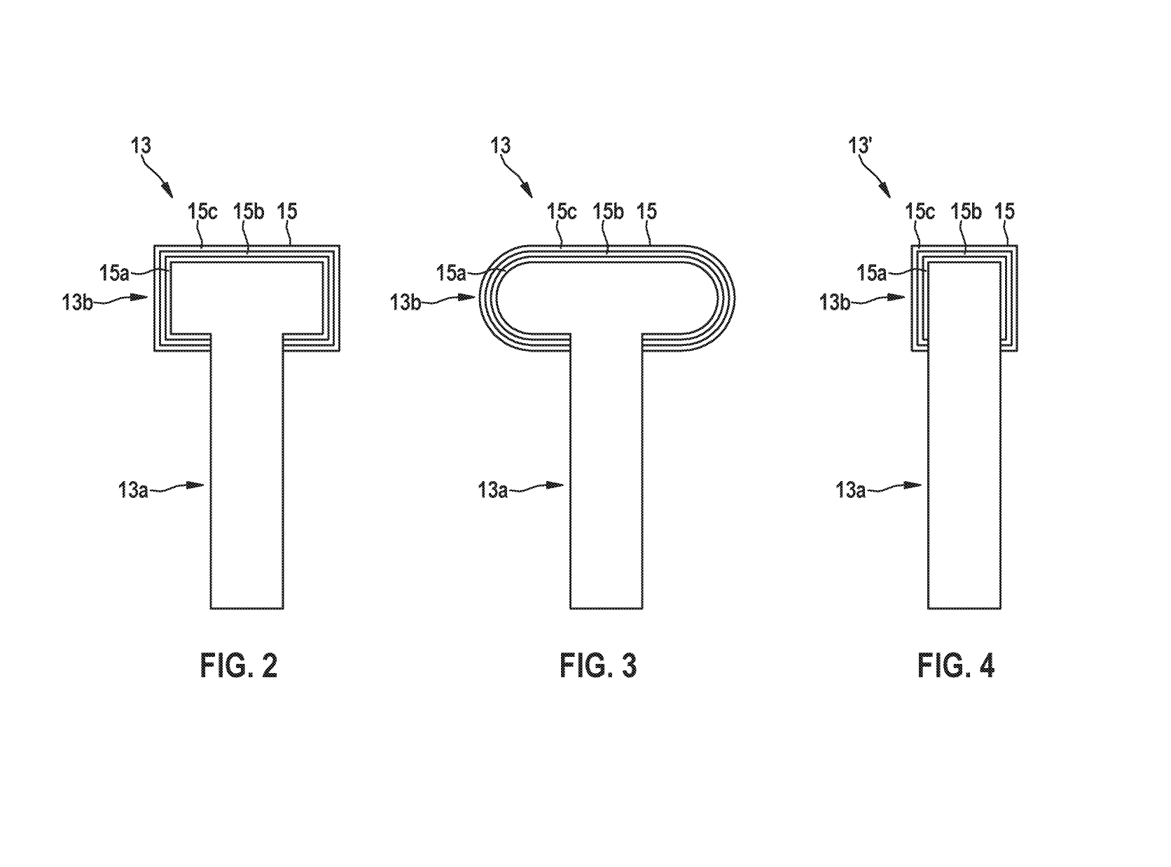

In all of FIGS. 2 to 7 the connection pin is denoted uniformly in each case, following on from FIG. 1 and regardless of the deviating geometric forms, by numerals 13 or 13′, and the referencing of individual portions or the corresponding coating is selected accordingly.

FIG. 2 shows a connection pin 13 with a main body 13a that is cylindrical over the majority of its extent and with an end 13b that is thickened in a stepped manner and is also cylindrical. A coating 15 is applied to the entire surface of the thickened end 13b, which coating comprises a lower adhesion promoter layer 15a, a solder connection layer 15b covering this, and an anti-oxidation layer 15c disposed there above. By contrast, no coating is shown on the periphery of the longer pin portion 13a with smaller diameter; as appropriate, a coating promoting the brazing in a feedthrough can be provided here, however this coating is irrelevant in the context of the present invention.

FIG. 3 shows a further connection pin 13 with a very similar structure to that shown in FIG. 2, although it differs therefrom by a thickened end 13b rounded in a balloon-like manner. The structure of the coating is the same as in FIG. 2.

FIG. 4 shows, as a further example, a connection pin 13′, which comprises a cylindrical body 13a with a constant diameter between both ends. At one of the ends a coating 15 covering the end face and part of the periphery of the main body 13a is provided, the structure of said coating corresponding to that of the coatings from FIGS. 2 and 3. Due to the multi-part coating, a thickening of the connection pin is provided at the coated end, however the dimensions of said thickening are much smaller than those in the case of the connection pins according to FIGS. 2 and 3.

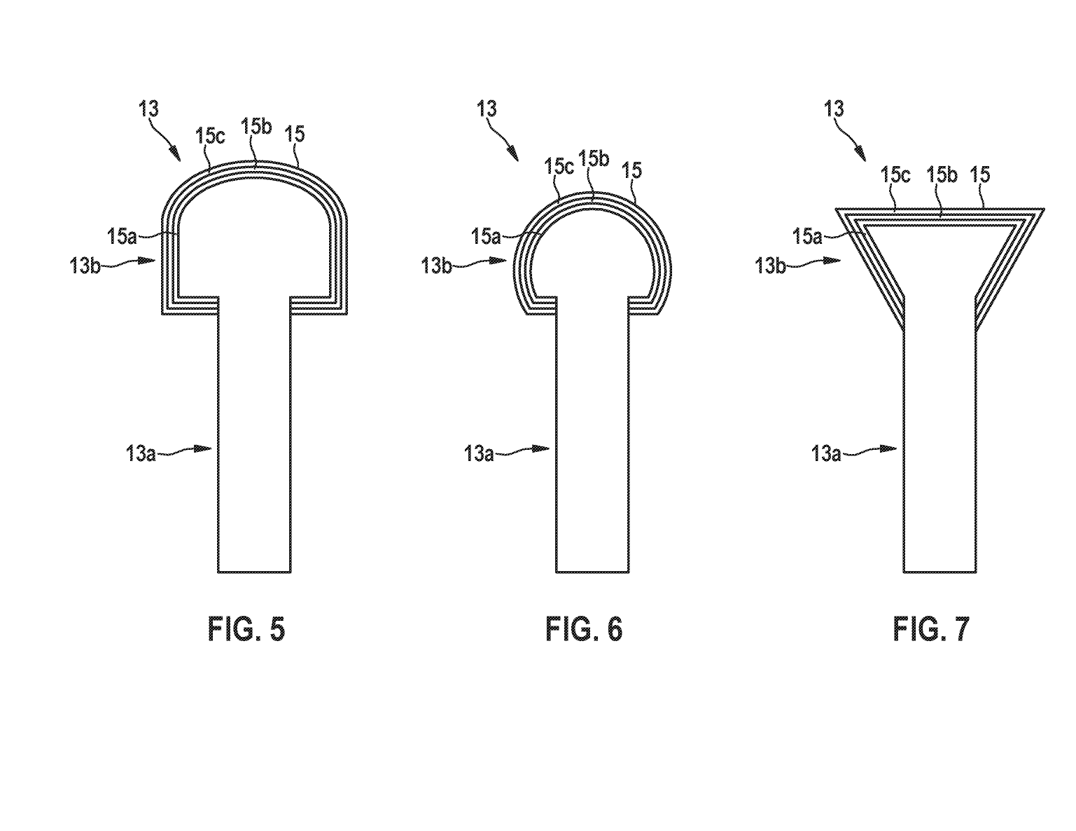

FIG. 5 shows, as a further example, a connection pin 13, in which again—as in FIGS. 2 and 3—at the end of the cylindrical portion 13a there is provided a stepped integral thickening 13b made of the material of the connection pin body. Here, this has the form of a cylindrical portion with spherical free end face. The structure of the coating 15 corresponds to the previously mentioned examples.

FIG. 6 shows, as a further example, a connection pin 13 with fundamentally the same structure as FIGS. 2, 3 and 5, but with a thickened end portion 13b in the form of a sphere segment which is slightly larger than a hemisphere.

FIG. 7 lastly shows, as a further exemplary embodiment, a connection pin 13, of which the larger cylindrical part 13a widens conically into a truncated cone-shaped end 13b. Otherwise, the structure here is again the same as in the aforementioned embodiments.

The invention can be provided in many other variants and in a wide range of combinations of the above features presented as features of different embodiments, in particular also with more than two-part or two-layer coating systems, with materials other than those mentioned above, and in many geometric designs of the respective thicknesses and regions of smaller diameter remote from the end.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

1. A connection pin for electrical connection of a cable mount, the connection pin comprising:
a thickened end for an integrally bonded connection to a conductor surface of the cable mount;
at least a portion of a surface of said thickened end having a solderable coating with at least two functionally differentiated sub-layers arranged above one another, wherein one of said sub-layers is a solder connection layer with an anti-oxidation layer provided above said solder connection layer and/or an adhesion promoter layer provided beneath said solder connection layer.

2. The connection pin according to claim 1, wherein said coating is disposed on a connection pin body and comprises said adhesion promoter layer and, at least indirectly thereabove, said solder connection layer and, at least indirectly thereabove, said anti-oxidation layer.

3. The connection pin according to claim 1, wherein said coating covers an entire surface of said thickened region.

4. The connection pin according to claim 1, wherein said thickened end has a base and a periphery, and said coating covers only said base and optionally said periphery or portions of said periphery of said thickened end.

5. The connection pin according to claim 1, wherein said solder connection layer comprises at least one metal selected from the group consisting of gold, platinum, copper, silver, nickel, iridium and palladium, and said adhesion promoter layer comprises at least one metal selected from the group consisting of palladium, tungsten, platinum and tantalum or at least one compound selected from the group consisting of niobium nitride, titanium nitride, copper nitride and nickel nitride.

6. The connection pin according to claim 2, wherein said connection pin body is formed from a non-precious metal and a layer of the coating directly contacting said non-precious metal is formed as an adhesion promoter layer.

7. The connection pin according to claim 6, wherein said adhesion promoter layer comprises at least one metal selected from the group consisting of palladium, tungsten, platinum and tantalum or at least one compound selected from the group consisting of niobium nitride, titanium nitride, copper nitride and nickel nitride.

8. The connection pin according to claim 1, wherein said coating, or at least a layer of said coating, is embodied as a galvanic coating or as a layer having the characteristics of a layer produced by a vacuum coating method.

9. The connection pin according to claim 1, with at least one portion which has a sub-layer comprising a biocompatible material.

10. The connection pin according to claim 9, wherein said biocompatible material is selected from the group consisting of titanium, tantalum, niobium, gold, platinum, palladium and an alloy of at least one of these metals.

11. The connection pin according to claim 1, wherein said thickened end of the connection pin has an extension portion formed as a cylinder, truncated cone or sphere portion or as a square or polygonal prism or square or multi-sided truncated pyramid, substantially in the form of a nail head.

12. A feedthrough of an implantable electro-medical device, the feedthrough comprising at least one connection pin according to claim 1.

13. The feedthrough according to claim 12, wherein the electro-medical device is a cardiac pacemaker or a cardioverter.
